(19) United States
(12) Patent Application Publication
(10) Pub. No.: US 2025/0216387 A1
(43) Pub. Date: Jul. 3, 2025

(54) INDEXED LATERAL FLOW MICROARRAY DEVICE

(71) Applicant: QuantiScientifics, LLC, Orange, CA (US)

(72) Inventor: Robert Stanley Matson, Orange, CA (US)

(73) Assignee: QuantiScientifics, LLC, Orange, CA (US)

(21) Appl. No.: 18/999,993

(22) Filed: Dec. 23, 2024

Related U.S. Application Data
(60) Provisional application No. 63/616,203, filed on Dec. 29, 2023.

(51) Int. Cl. G01N 33/543

(52) U.S. Cl. CPC G01N 33/54388

(57) ABSTRACT

The present invention relates to an indexed lateral flow microarray device (iLFM) for direct visual detection of multiple analytes in biological samples. The device utilizes indexing elements to eliminate the need for imaging scanners or image analysis software typically required for lateral flow strips. As a result, users can easily identify multiple analytes in a biological sample by visually inspecting analyte spots aligned with a set of positional reference markers. The iLFM features a substrate, such as a nitrocellulose strip, embedded with specific analyte affinity capture elements arranged in a Cartesian (x,y) array format simplifying the analysis process, enhancing accessibility, and expanding the potential applications of lateral flow assays in various diagnostic settings. By enabling a direct visual method for detecting multiple analytes, the iLFM has significant potential in areas like clinical diagnostics, environmental monitoring, and food safety, making it a promising tool for rapid and efficient analyte detection.

### CLAIM FOR PRIORITY

This application claims priority from a prior utility provisional application with application No. 63/616,203 filed on Dec. 29, 2023. The entire collective teachings thereof are herein incorporated by reference.

### BACKGROUND OF THE INVENTION

Embodiments of this disclosure generally relate to the field of diagnostic devices and methods, and more particularly, to an indexed lateral flow microarray device for simultaneous detection, quantification, and analysis of multiple analytes in a single sample.

Lateral flow immunoassays (LFAs) are a straightforward and widely used diagnostic tool for detecting specific analytes in liquid samples, often requiring just one or two steps for qualitative analysis. These tests, also known as lateral flow tests, dipsticks, or strip tests, involve a test membrane with capture reagents applied in specific lines or zones. These reagents are designed to bind to target analytes if present in the sample. When a liquid sample is introduced to the membrane, capillary action draws it along the length of the strip. As the sample travels, analytes in the sample interact with the capture reagents, leading to measurable changes within the test zones, which allow for easy detection of the target analytes. This mechanism is commonly used for tests like pregnancy tests, infectious disease detection, and environmental monitoring due to its simplicity, speed, and ease of use.

The current technology used in art diagnostics, especially within lateral flow immunoassays (LFAs), presents challenges for reliable and immediate visual interpretation of results. Relying on external imaging devices can add complexity, raising both operational costs and the need for specialized personnel. This dependence not only reduces accessibility but also introduces variability in interpreting results, as factors like lighting, camera quality, and user experience can all impact consistency. To address these limitations, developing more robust and self-contained visualization methods for LFAs could provide a clearer and more consistent “at-a-glance” result, reducing reliance on external devices and enhancing reliability for a broader user base.

In multi-analyte testing with lateral flow immunoassays (LFAs), efforts to visually identify multiple analytes in a sample without external equipment have indeed faced challenges. While detection reagents can indicate the presence of analytes through visible spots or lines on the test membrane, reliably interpreting these results can be problematic. For instance, U.S. Pat. No. 10,996,221 illustrates a multi-analyte detection approach, yet it highlights issues in achieving a clear interpretation of results by using control or reference elements. Similarly, applications like PCT Appl. No. 2015008287 and U.S. Pat. Appl. No. 20180319657 discloses methods for detecting and analyzing multiple analytes in LFAs. However, none of these methods effectively use any systematic matrix or way of using control elements for a better result appearance. The visual clarity remains limited due to factors like reagent variability and overlapping signals, underscoring a need for innovation in self-interpreting LFA designs that could enhance usability and accessibility for point-of-care testing and self-diagnosis.

Accordingly, it is apparent that a need exists for a microarray device that enables clear, at-a-glance interpretation of results by organizing each analyte detection zone in a distinct and indexed format. Such a device has the potential to streamline the diagnostic process and broaden the reach of lateral flow assays, enhancing their effectiveness in fields ranging from healthcare to environmental monitoring.

### BRIEF SUMMARY OF THE INVENTION

The present invention relates to a device designed for conducting a multiplex lateral flow immunoassay. The device allows for the simultaneous testing of a liquid sample, such as a biological sample, for multiple analytes of interest. Additionally, methods are provided that utilize the device for the concurrent detection of several analytes within a liquid test sample.

The indexed lateral flow microarray device (iLFM) of the present disclosure represents a significant advancement in the field of diagnostic testing by enabling direct visual identification of multiple analytes in biological samples. Unlike traditional lateral flow assays that require complex imaging systems or software for analysis, the iLFM simplifies the detection process. Users can readily observe the results by examining the positions of analyte spots against a set of clearly defined positional reference markers. This streamlined approach not only enhances usability but also improves the speed of analysis, making it particularly valuable in settings where rapid results are essential.

In one aspect, the present invention provides an indexed lateral flow microarray (iLFM) device for detecting multiple analytes in a biological sample, comprising: a substrate, which includes a sample pad, a conjugate pad, and a wicking pad; a plurality of analyte affinity capture elements arranged on the substrate in a Cartesian (x, y) array format, each capture element capable of binding a specific analyte from the biological sample; a set of indexing elements arranged on the substrate, comprising: indexing control elements which are visible alignment marker spots that define the y-coordinates associated with the arrayed capture elements; indexing array elements which are alignment spots that define the x-coordinates of the array. The intersection of the y-coordinate defined by the indexing control element and the x-coordinate defined by the indexing array element corresponds to a specific analyte spot within the array, binding events occurring at the intersections produce a visually detectable signal that indicates the presence of a bound analyte.

Another aspect of the invention provides a method for detecting multiple analytes in a biological sample, the method comprising: (a) providing an indexed lateral flow microarray (iLFM) device comprising, a substrate that comprises a sample pad, a conjugate pad, and a wicking pad; a plurality of analyte affinity capture elements arranged on the substrate in a Cartesian (x, y) array format, each capture element is capable of binding a specific analyte from the biological sample; a set of indexing elements arranged on the substrate, comprising: indexing control elements which are visible alignment marker spots that define y-coordinates associated with the capture elements, indexing array elements which are alignment spots that define the x-coordinates associated with the capture elements; (b) applying the biological sample to the sample pad of the iLFM device; (c) allowing the biological sample to pass through the conjugate pad, the plurality of analyte affinity capture elements, and the set of indexing elements arranged on the substrate using the wicking pad; (d) determining the intersection of y-coordinate defined by the indexing control element and x-coordinate defined by the indexing array element to locate a specific analyte spot within the array; and (e) detecting binding events occurring at the intersection, the presence of a bound analyte is indicated by a visually detectable signal.

At the core of the iLFM's design is the substrate, typically a backed nitrocellulose strip, which serves as the platform for the assay. The substrate may be a backed nitrocellulose strip or another porous material. This substrate is embedded with defined analyte affinity capture elements arranged in a Cartesian (x,y) array format. The organized layout allows for precise targeting of specific analytes, ensuring that each analyte has a dedicated location on the strip. This structure enhances the reliability of the assay by minimizing cross-reactivity and ensuring that the binding of the analyte is accurately captured.

To facilitate the detection process, the iLFM device includes the set of indexing elements. These elements are crucial for confirming the presence or absence of an analyte based on their physical (x) coordinate registration with the indexing elements. Each analyte's location on the strip corresponds to a specific index, enabling users to quickly ascertain which analytes are present in the sample. The clear mapping of analyte locations to specific coordinates simplifies the interpretation of results, making it accessible even for users with limited technical expertise.

In addition to the indexing elements, the iLFM device incorporates indexing control elements that provide further precision in the assay. The indexing control elements help to co-locate the capture elements through registration in the (y) coordinate. This dual-coordinate system not only enhances the accuracy of analyte detection but also serves as a quality control measure. By verifying the successful flow of the analyte across the arrayed capture elements, these controls ensure that the assay functions correctly, providing confidence in the results obtained.

In yet another aspect of the invention, the indexed lateral flow microarray (iLFM) device and its associated method for detecting multiple analytes in a biological sample include the following criteria for interpreting results: a positive result is indicated by the appearance of a signal on both the capture elements comprising food protein allergen spots and the indexing spots; a negative result is indicated by signal development occurring only on the indexing spots; and an invalid test result is identified when the indexing spots are absent.

In another aspect of the invention, a kit for detecting multiple analytes using the indexed lateral flow microarray device, the kit comprising: the indexed lateral flow microarray device; and instructions for using the device to detect and visually identify the analytes based on their indexed x, and y coordinates.

The disclosed method provides a novel approach for detecting multiple analytes, where the appearance of signals on both capture and indexing elements confirms analyte presence. This approach allows rapid, reliable results, particularly suited for field applications requiring immediate decision-making. Ultimately, the indexed lateral flow microarray device offers a robust and user-friendly solution for multi-analyte detection in biological samples. Its innovative design combines a straightforward visual readout with a structured approach to analyte capture and identification. As a result, the iLFM is well-suited for diverse applications, from clinical diagnostics to field testing, where rapid and accurate detection of multiple analytes is essential. This technology paves the way for more efficient diagnostic practices, bridging the gap between complex analytical methods and practical, real-world testing environments.

The indexed lateral flow microarray device (iLFM) of the present disclosure enables a visual determination of multiple analytes on a single rapid test strip without the need for electronic instrumentations such as rapid strip readers or image analysis software. Higher levels of multiplexing are possible based upon the number of indexing elements than possible with line-based rapid test strips. For instance, 5 x-indexing alignment markers with 3 y-indexing markers can define 15 analytes in an array, while 5×5 indexing markers would yield 25 visually identifiable analytes confined to a standard lateral flow strip. Thus, multiple analytes in a biological sample can be identified by a simple visual read of analyte spots that are registered against a set of positional reference markers. The use of indexing markers not only enhances the clarity of results but also plays a critical role in establishing the credibility of the assay. By effectively binding to the immuno-complex and providing a reference point for visual identification, the indexing markers contribute to the overall robustness of the iLFM device. This design ultimately allows healthcare providers to confidently interpret the results, facilitating timely decision-making in clinical settings. The indexing markers are a vital component of the indexed lateral flow microarray device, enabling precise identification of sIgE antibodies in human plasma through their strategic design and function. Their incorporation into the assay enhances both the sensitivity and specificity of the detection process, ensuring that the assay is not only effective in identifying potential food allergens but also reliable in providing clear, interpretable results.

The embodiments covered by this patent are defined by the claims. The summary above provides a general overview of various aspects and introduces some of the concepts that are discussed in greater detail in the following description section. This summary is not meant to identify the key or essential features of the claimed subject matter, nor is it intended to be used on its own to determine the scope of the claims. The subject matter should be understood with reference to the entire specification, including any relevant drawings and the claims themselves.

### BRIEF DESCRIPTION OF THE DRAWINGS

The embodiments herein will be better understood from the following detailed description with reference to the drawings, in which:

FIG. 1 illustrates an indexed lateral flow microarray device for detecting multiple analytes in a biological sample according to various embodiments of the present disclosure.

FIG. 2 illustrates the indexed microarray of FIG. 1 showing the use of indexing control elements and indexing array elements to identify specific analytes within the array according to various embodiments of the present disclosure.

FIG. 3 illustrates a fully developed multiplex lateral flow immunoassay of FIG. 1 according to various embodiments of the present disclosure.

FIG. 4 illustrates three distinct positive analyte spots among the eight available in the indexed microarray of FIG. 1 according to various embodiments of the present disclosure.

FIG. 5 illustrates an immunoassay performed using the indexed microarray of FIG. 1 according to various embodiments of the present disclosure.

FIG. 6 illustrates the interpretation of assay results of the immunoassay performed using the indexed microarray of FIG. 5 according to various embodiments of the present disclosure.

FIGS. 7A-B illustrate flow charts describing a method for detecting multiple analytes in a biological sample according to various embodiments of the present disclosure.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims. An indexed lateral flow microarray device is discussed herein. In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below. The present invention will now be described by referencing the appended figures representing preferred embodiments.

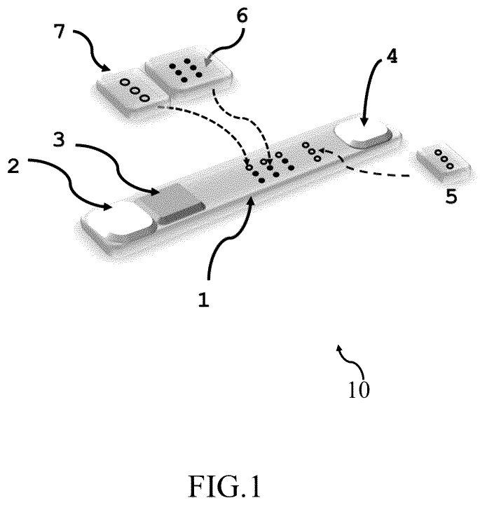

1. FIG. 1 illustrates an indexed lateral flow microarray device for detecting multiple analytes in a biological sample according to various embodiments of the present disclosure. The indexed lateral flow microarray device 10 includes a substrate (1) serving as a platform. The substrate (1) may be a backed nitrocellulose strip or another porous material. The substrate (1) includes a sample pad (2), a conjugate pad (3), and a wicking pad (4). The sample pad (2) serves as the initial contact point for a liquid sample, typically the biological sample such as human plasma. The sample pad (2) is designed to facilitate the efficient absorption and migration of the sample into subsequent sections of the substrate (1). Positioned downstream from the sample pad (2), the conjugate pad (3) includes gold particle conjugates that are linked to specific a anti-immunoglobulin, such as mouse anti-IgE. When the sample reaches the conjugate pad (3), the specific analytes within the sample, such as sIgE antibodies, bind to the gold particle anti-immunoglobulin conjugates forming an “immuno-complex” that will later interact with the capture elements on the substrate (1). The anti-immunoglobulins include anti-IgA, anti-IgM, anti-IgG, anti-IgE derived from any host species, such as mouse, goat, donkey, or chicken. The wicking pad (4) serves to draw the sample through capillary action, promoting the flow of the sample from the sample pad (2) through the conjugate pad (3) and to a plurality of capture elements (6) arranged on the substrate (1) in a Cartesian (x, y) array format. Each capture element is capable of binding a specific analyte from the biological sample. The movement of the sample is crucial ensuring the sample interacts effectively with the capture elements (6). The capture elements (6) include multiple capture spots, each containing antibodies specific to various analytes of interest. The array format allows for the simultaneous detection of multiple targets within a single sample, significantly enhancing the assay's multiplexing capabilities. The device includes a set of indexing elements (5,7) arranged on the substrate (1). The set of indexing elements include antibody recognizing immunoglobulins such as mouse IgG which captures the gold particle anti-immunoglobulin conjugates. The indexing control elements (5) are visible alignment marker spots that define y-coordinates associated with the capture elements (6). The indexing array elements (7) are alignment spots that define the x-coordinates associated with the capture elements (6). An intersection of the x-coordinate and y-coordinate indexing elements defines specific locations for detecting the presence of the analytes in the biological sample. Binding events occurring at the intersections produce a visually detectable signal that indicates the presence of a bound analyte.

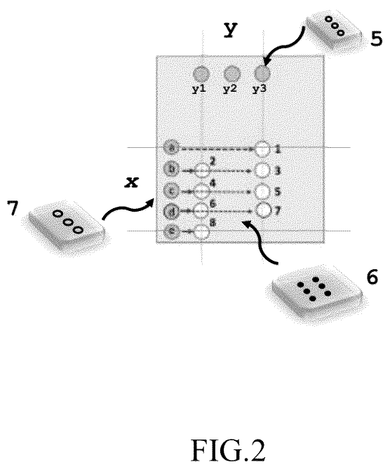

FIG. 2 illustrates the indexed microarray of FIG. 1 showing the use of indexing control elements and indexing array elements to identify specific analytes within the array according to various embodiments of the present disclosure. As shown in FIG. 2, the indexing control elements (5) are visible alignment marker spots that establish the y-coordinates associated with the capture elements (6). They serve as reference points that facilitate the accurate identification of specific capture spots on the substrate (1). The indexing array elements (7) which are alignment spots that define the x-coordinates of the capture elements (6), work in conjunction with the indexing control elements (5) to provide a clear coordinate system for locating each analyte spot. Positioned along the vertical axis, the indexing control elements (5) provide reference points that enhance the spatial organization of the array as depicted in FIG. 2. Each indexing array element (7) corresponds to a specific column of capture elements, working in tandem with the indexing control elements (5). This grid-like structure allows for precise coordinate registration, enabling users to pinpoint the location of specific analyte spots quickly. The combination of these two indexing systems: y-coordinates defined by the indexing control elements (5) and x-coordinates defined by the indexing array elements (7) results in a clear and organized coordinate grid. The intersection of these coordinates allows users to accurately identify the location of each analyte spot. For example, the intersection denoted as (y3, xa) would correspond to analyte spot #1 within the array.

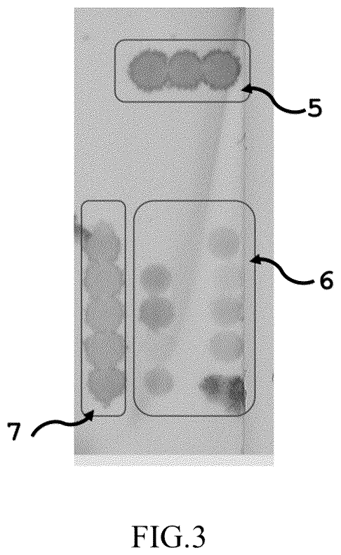

FIG. 3 illustrates a fully developed multiplex lateral flow immunoassay of FIG. 1 according to various embodiments of the present disclosure. The array consists of multiple capture spots where protein allergen extracts are printed in a 2×5 format. This arrangement allows for the simultaneous testing of various allergens, maximizing the assay's multiplexing capabilities. Each spot is tailored to capture specific immunoglobulins, enabling the detection of different allergenic proteins present in a biological sample, such as human plasma. The vertical alignment of the array is achieved using the three indexing control elements (5). These visible markers serve as reference points that define the y-coordinates of the arrayed capture elements. Their strategic placement ensures that the rows of capture elements are clearly delineated, facilitating easy navigation and interpretation during result analysis. The horizontal alignment of the array is established using the five indexing array elements (7). These alignment markers define the x-coordinates of the capture elements, creating a systematic grid structure. This x-direction alignment complements the y-direction markers, ensuring that each capture element can be accurately located based on its coordinates. The immunoassay results in a total of eight analyte spots that have developed at varying levels of signal intensity. Each signal indicates the presence of specific analytes within the biological sample, reflecting the binding interactions between the sample's immunoglobulins and the protein allergens immobilized on the array. The variation in signal intensity provides valuable quantitative information regarding the concentration of specific allergens present in the sample. The positive spots are identified through a visual readout that leverages the indexing elements integrated into the device. Each positive analyte spot is clearly marked and located based on the established coordinate system, derived from the indexing control elements (5) and the indexing array elements (7).

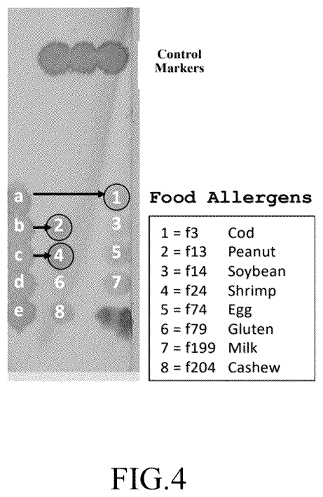

FIG. 4 illustrates three distinct positive analyte spots among the eight available in the indexed microarray of FIG. 1 according to various embodiments of the present disclosure. These positive spots indicate the presence of IgE antibodies specific to food allergens in the human plasma. The remaining five spots may not exhibit any signal due to the absence of corresponding analytes in the sample, underscoring the selective nature of the assay. It represents an example of the results obtained from the multiplex lateral flow immunoassay conducted on a patient plasma sample. In this illustration, only three out of the eight potential analyte spots have developed, highlighting the assay's capability to detect specific targets while demonstrating the use of indexing elements (5,7) for clear visual interpretation. The presence of the three positive spots, along with their locations indicated by the indexing system, allows for a quick assessment of the patient's allergenic profile.

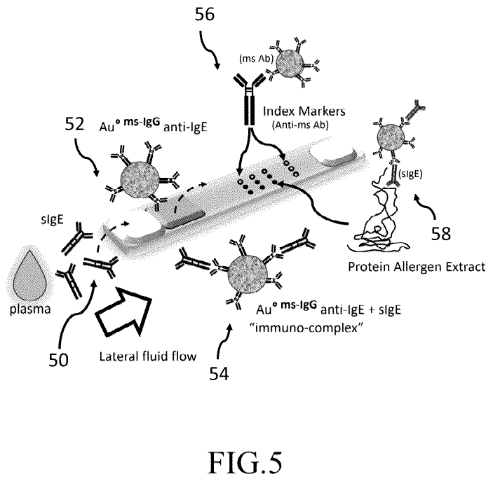

FIG. 5 illustrates an immunoassay performed using the indexed microarray of FIG. 1 according to various embodiments of the present disclosure. The immunoassay is fundamentally based on the binding interaction between food allergen-specific human sIgE antibodies present in the human plasma and the various components of the assay. As depicted in FIG. 5, food protein extracts are systematically printed onto the substrate (1) including a nitrocellulose strip in an organized array format forming the capture elements (6). Upon application of human plasma containing sIgE antibodies to the sample pad (1), a critical flow process is initiated, guiding the sample toward the conjugate pad (3). At this stage, the sIgE antibodies [50] present in the plasma bind to the conjugate composed of gold particles and mouse anti-IgE antibodies [52]. This interaction is essential for the formation of a stable “immuno-complex” [54], which subsequently migrates along the substrate (1). As the formed immuno-complex [54] travels upward through the substrate (1), it encounters the indexing markers (5, 7). Here, the anti-mouse antibody [56], which is incorporated into the indexing markers, binds to the mouse IgG component of the immuno-complex [54]. This binding secures the immuno-complex to both the indexing markers and the food protein allergens present in the array as capture elements (6), facilitated by the specific interaction of sIgE antibodies bound to the immuno-complex [54] with the respective allergens [58] printed on the substrate (1).

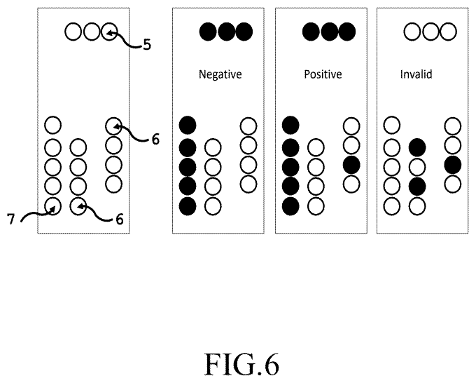

FIG. 6 illustrates the interpretation of assay results of the immunoassay performed using the indexed microarray of FIG. 5 according to various embodiments of the present disclosure. The visualization of results in the assay is streamlined through the unique indexing system. A positive result (FIG. 6A) is confirmed when a visible signal appears on both the capture elements (6) including food protein allergen spots and the indexing elements (5, 7). This indicates that the sample has successfully produced an immuno-complex that has bound to the specific allergens and been captured at the indexing markers. In contrast, a negative result (FIG. 6B) is indicated by signal development solely on the indexing spots, suggesting that no specific sIgE antibodies have interacted with the allergens in the array. This is critical for confirming the absence of food allergens in the tested plasma sample. Additionally, an invalid test result (FIG. 6C) is noted when the indexing spots are entirely absent from the assay output. This scenario may suggest a malfunction within the assay process, indicating that the sample may not have migrated properly through the device or that other procedural errors occurred. Thus, the indexing markers serve as essential controls for both confirming the presence of specific analytes and ensuring the reliability of the test results.

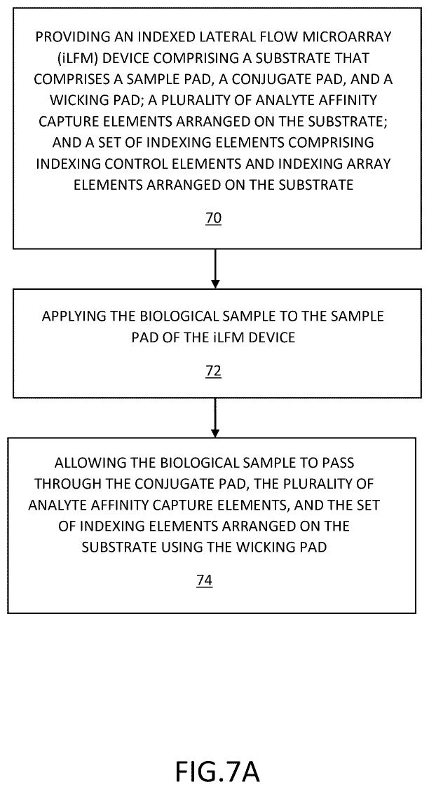

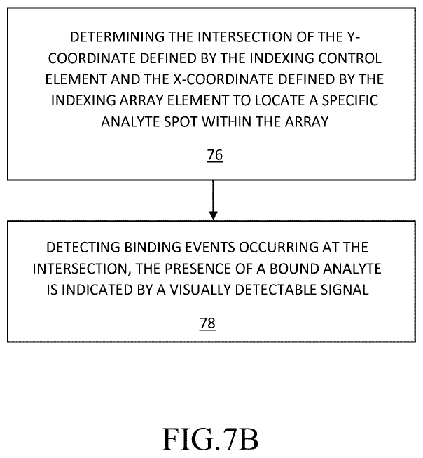

FIGS. 7A-B illustrate flow charts describing a method for detecting multiple analytes in a biological sample according to various embodiments of the present disclosure. The method includes at step 70, providing an indexed lateral flow microarray (iLFM) device comprising, a substrate that comprises a sample pad, a conjugate pad, and a wicking pad; a plurality of analyte affinity capture elements arranged on the substrate in a Cartesian (x, y) array format, wherein each capture element is capable of binding a specific analyte from the biological sample; a set of indexing elements arranged on the substrate, comprising: indexing control elements which are visible alignment marker spots that define y-coordinates associated with the capture elements, indexing array elements which are alignment spots that define the x-coordinates associated with the capture elements. The method includes at step 72, applying the biological sample to the sample pad of the iLFM device. The method includes at step 74, allowing the biological sample to pass through the conjugate pad, the plurality of analyte affinity capture elements, and the set of indexing elements arranged on the substrate using the wicking pad. The method includes at step 76, determining the intersection of the y-coordinate defined by the indexing control element and the x-coordinate defined by the indexing array element to locate a specific analyte spot within the array. The method includes at step 78, detecting binding events occurring at the intersection, wherein the presence of a bound analyte is indicated by a visually detectable signal.

The Indexed Lateral Flow Microarray Device (iLFM) of the present disclosure represents a promising innovation in simplifying multi-analyte detection with LFAs while prioritizing accuracy and reliability. By integrating indexing and control elements directly onto the test membrane, the iLFM design allows users to visually detect analytes without needing complex imaging systems. This approach minimizes reliance on external equipment and reduces interpretation errors, making LFAs more user-friendly and accessible for various diagnostic applications. The iLFM's unique structure enables clear, at-a-glance interpretation of results by organizing each analyte detection zone in a distinct and indexed format. This structured layout not only aids in distinguishing multiple analytes in a single test but also provides quality control elements to confirm test validity. Overall, the iLFM has the potential to streamline the diagnostic process and broaden the reach of lateral flow assays, enhancing their effectiveness in fields ranging from healthcare to environmental monitoring.

In closing, it is to be understood that the embodiments of the invention disclosed herein are illustrative of the principles of the present invention. Other modifications that may be employed are within the scope of the invention. Thus, by way of example, but not of limitation alternative configurations of the present invention may be utilized in accordance with the teachings herein. Accordingly, the present invention is not limited to that precisely as shown and described.

What is claimed is:

1. An indexed lateral flow microarray (iLFM) device for detecting multiple analytes in a biological sample, comprising:
a substrate that comprises a sample pad, a conjugate pad, and a wicking pad;
a plurality of analyte affinity capture elements arranged on the substrate in a Cartesian (x, y) array format, wherein each capture element is capable of binding a specific analyte from the biological sample;
a set of indexing elements arranged on the substrate, comprising:
indexing control elements, wherein the control elements are visible alignment marker spots that define y-coordinates associated with the capture elements;
indexing array elements, wherein the array elements are alignment spots that define the x-coordinates associated with the capture elements,
wherein an intersection of the y-coordinate defined by the indexing control element and the x-coordinate defined by the indexing array element corresponds to a specific analyte spot within the array, wherein binding events occurring at the intersections produce a visually detectable signal that indicates the presence of a bound analyte.

2. The microarray device of claim 1, wherein the conjugate pad comprises gold particle anti-immunoglobulin conjugates, wherein the analytes in the biological sample bind to the gold particle anti-immunoglobulin conjugates forming an “immuno-complex” to interact with the capture elements.

3. The microarray device of claim 2, wherein the set of indexing elements comprise antibody recognizing immunoglobulins which captures the gold particle anti-immunoglobulin conjugates.

4. The microarray device of claim 1, wherein the indexing control elements and indexing array elements collectively form a grid-like coordinate system that enables precise identification of the specific analyte spot within the array.

5. The microarray device of claim 1, wherein the capture elements are selected from a group comprising proteins, allergens, nucleic acids, drugs or other biomolecules.

6. The microarray device of claim 1, wherein the substrate is a porous nitrocellulose substrate.

7. A method for detecting multiple analytes in a biological sample, the method comprises:
providing an indexed lateral flow microarray (iLFM) device comprising,
a substrate that comprises a sample pad, a conjugate pad, and a wicking pad;
a plurality of analyte affinity capture elements arranged on the substrate in a Cartesian (x, y) array format, wherein each capture element is capable of binding a specific analyte from the biological sample;
a set of indexing elements arranged on the substrate, comprising:
indexing control elements, wherein the control elements are visible alignment marker spots that define y-coordinates associated with the capture elements,
indexing array elements, wherein the array elements are alignment spots that define the x-coordinates associated with the capture elements;
applying the biological sample to the sample pad of the iLFM device;
allowing the biological sample to pass through the conjugate pad, the plurality of analyte affinity capture elements, and the set of indexing elements arranged on the substrate using the wicking pad;
determining the intersection of the y-coordinate defined by the indexing control element and the x-coordinate defined by the indexing array element to locate a specific analyte spot within the array; and
detecting binding events occurring at the intersection, wherein the presence of a bound analyte is indicated by a visually detectable signal.

8. The method of claim 7, wherein the method comprises forming an “immuno-complex” in the conjugate pad, wherein the conjugate pad comprises gold particle anti-immunoglobulin conjugates, wherein the analytes in the biological sample binds to the gold particle anti-immunoglobulin conjugates forming the “immuno-complex” to interact with the capture elements.

9. The method of claim 8, wherein the set of indexing elements comprise antibody recognizing immunoglobulins which captures the gold particle anti-immunoglobulin conjugates.

10. The method of claim 7, wherein the indexing control elements and indexing array elements collectively form a grid-like coordinate system that enables precise identification of the specific analyte spot within the array.

11. The method of claim 7, wherein the capture elements are selected from a group comprising proteins, allergens, or other biomolecules.

12. The method of claim 7, wherein the substrate is a porous nitrocellulose substrate.

13. A kit for detecting multiple analytes in a biological sample, the kit comprising:
the indexed lateral flow microarray device of claim 1;
and instructions for using the device to detect and visually identify the analytes based on their indexed x, and y coordinates.
